(19) United States
(12) Patent Application Publication
(10) Pub. No.: US 2022/0192487 A1
(43) Pub. Date: Jun. 23, 2022

(54) METHOD AND SYSTEM FOR DETERMINING DOMINANT EYE, AND NON-TRANSITORY COMPUTER-READABLE RECORDING MEDIUM

(71) Applicant: VTOUCH CO., LTD., Seoul (KR)

(72) Inventors: Seok Joong KIM, Seoul (KR); Jik Han JUNG, Yongin-si (KR)

(73) Assignee: VTOUCH CO., LTD., Seoul (KR)

(21) Appl. No.: 17/685,985

(22) Filed: Mar. 3, 2022

Related U.S. Application Data
(63) Continuation of application No. PCT/KR2020/011269, filed on Aug. 24, 2020.

(30) Foreign Application Priority Data
Sep. 4, 2019 (KR) 10-2019-0109479
Oct. 8, 2019 (KR) 10-2019-0124862

(51) Int. Cl. A61B 3/113; A61B 3/00; A61B 3/02; G06F 3/01; A61B 3/14

(52) U.S. Cl. CPC A61B 3/113; A61B 3/0033; A61B 3/14; A61B 3/02; G06F 3/013; A61B 3/0041

(57) ABSTRACT

A method of determining a dominant eye includes acquiring indicator coordinates, left-eye coordinates, and right-eye coordinates of a user at a plurality of time points; calculating left-eye difference information about an angle, distance, or direction specified based on a straight line passing through the indicator coordinate and the left-eye coordinate at a first time point and a straight line passing through the indicator coordinate and the left-eye coordinate at a second time point, and right-eye difference information about an angle, distance, or direction specified based on a straight line passing through the indicator coordinate and the right-eye coordinate at the first time point and a straight line passing through the indicator coordinate and the right-eye coordinate at the second time point; and determining the dominant eye of the user with reference to the left-eye difference information and the right-eye difference information.

### CROSS-REFERENCE TO RELATED APPLICATION

This application is a continuation application of Patent Cooperation Treaty (PCT) International Application No. PCT/KR2020/011269 filed on Aug. 24, 2020, which claims priority to Korean Patent Application No. 10-2019-0109479 filed on Sep. 4, 2019, and Korean Patent Application No. 10-2019-0124862 filed on Oct. 8, 2019. The entire contents of PCT International Application No. PCT/KR2020/011269, Korean Patent Application No. 10-2019-0109479 and Korean Patent Application No. 10-2019-0124862 are hereby incorporated by reference.

### TECHNICAL FIELD

The present disclosure relates to a method, a system and a non-transitory computer-readable recording medium for determining a dominant eye.

### BACKGROUND

In recent years, as interest in augmented reality (AR) or virtual reality (VR) is increasing and research and development in related technical fields are actively carried out, a variety of techniques for controlling an object using a user's body portion have been introduced.

As an example of a prior art associated with the above, there is Korean Registration Patent No. 1630153 that discloses an electronic apparatus including a first camera, a second camera, a microphone, a display, and a processor electrically connected to the first camera, the second camera, the microphone, and the display. The processor is configured to display a user interface (UI) including a plurality of objects on the display, obtain user's gaze information from the first camera, activate a first object corresponding to the user's gaze information among the plurality of objects, determine at least one input manner corresponding to a type of the activated first object among a gesture input obtained from the second camera and a voice input obtained by the microphone, and when the determined input manner is a manner applicable to the first object, execute a function corresponding to the input manner with respect to the first object while the first object is in the activation state.

However, according to the techniques introduced so far as well as the above-described prior art, object control is performed based on the user's gaze information without determining a dominant eye of the user. As a result, an error between a non-dominant eye and the dominant eye makes the object control inaccurate. In addition, in order to determine the dominant eye, the dominant eye is required to be set in advance depending on the user's input, or an additional device is required to specify the dominant eye. This causes a very cumbersome operation in terms of space, economy, or time.

In this connection, the inventors of the present disclosure propose a technique capable of simply and quickly determining a user's dominant eye based on coordinates of a user's body portion at a plurality of time points.

### SUMMARY

One object of the present disclosure is to solve all the above-described problems.

Another object of the present disclosure is to simply and quickly determine a dominant eye of a user based on coordinates of an indicator and both eyes of the user at a plurality of time points.

Yet another object of the present disclosure is to accurately determine a dominant eye determination by controlling a timing at which sensitive feedback associated with the dominant eye determination is provided.

Representative configurations of the present disclosure to achieve the above objects are described below.

According to an aspect of the present disclosure, there is provided a method of determining a dominant eye, comprising the steps of: acquiring indicator coordinates, left-eye coordinates, and right-eye coordinates of a user at a plurality of time points; calculating left-eye difference information about an angle, distance, or direction specified based on a straight line passing through the indicator coordinate and the left-eye coordinate at a first time point and a straight line passing through the indicator coordinate and the left-eye coordinate at a second time point, and right-eye difference information about an angle, distance, or direction specified based on a straight line passing through the indicator coordinate and the right-eye coordinate at the first time point and a straight line passing through the indicator coordinate and the right-eye coordinate at the second time point; and determining the dominant eye of the user with reference to the left-eye difference information and the right-eye difference information.

According to another aspect of the present disclosure, there is provided a method of determining a dominant eye, comprising the steps of: acquiring indicator coordinates, left-eye coordinates, and right-eye coordinates of a user at a plurality of time points; calculating left-eye difference information about an angle, distance, or direction specified based on a straight line passing through the indicator coordinate at a first time point and the indicator coordinate at a second time point, and a straight line passing through the indicator coordinate and the left-eye coordinate at the first time point, and right-eye difference information about an angle, distance, or direction specified based on a straight line passing through the indicator coordinate at the first time point and the indicator coordinate at the second time point, and a straight line passing through the indicator coordinate and the right-eye coordinate at the first time point; and determining the dominant eye of the user with reference to the left-eye difference information and the right-eye difference information.

According to another aspect of the present disclosure, there is provided a method of determining a dominant eye, comprising the steps of: acquiring indicator coordinates, left-eye coordinates, and right-eye coordinates of a user at a plurality of time points; calculating left-eye difference information about an angle, distance, or direction specified based on a straight line passing through the indicator coordinate at a first time point and the indicator coordinate at a second time point and a straight line passing through the indicator coordinate and the left-eye coordinate at the second time point, and right-eye difference information about an angle, distance, or direction specified based on a straight line passing through the indicator coordinate at the first time point and the indicator coordinate at the second time point and a straight line passing through the indicator coordinate and the right-eye coordinate at the second time point; and determining the dominant eye of the user with reference to the left-eye difference information and the right-eye difference information.

According to another aspect of the present disclosure, there is provided a system for determining a dominant eye, which includes: a coordinate acquisition unit configured to acquire indicator coordinates, left-eye coordinates, and right-eye coordinates of a user at a plurality of time points; a difference information calculation unit configured to calculate left-eye difference information about an angle, distance, or direction specified based on a straight line passing through the indicator coordinate and the left-eye coordinate at a first time point and a straight line passing through the indicator coordinate and the left-eye coordinate at a second time point, and right-eye difference information about an angle, distance, or direction specified based on a straight line passing through the indicator coordinate and the right-eye coordinate at the first time point and a straight line passing through the indicator coordinate and the right-eye coordinate at the second time point; and a dominant eye determination unit that determines the dominant eye of the user with reference to the left-eye difference information and the right-eye difference information.

According to another aspect of the present disclosure, there is provided a system for determining a dominant eye, which includes: a coordinate acquisition unit configured to acquire indicator coordinates, left-eye coordinates, and right-eye coordinates of a user at a plurality of time points; a difference information calculation unit configured to calculate left-eye difference information about an angle, distance, or direction specified based on a straight line passing through the indicator coordinate at a first time point and the indicator coordinate at a second time point and a straight line passing through the indicator coordinate and the left-eye coordinate at the first time point, and right-eye difference information about an angle, distance, or direction specified based on a straight line passing through the indicator coordinate at the first time point and the indicator coordinate at the second time point and a straight line passing through the indicator coordinate and the right-eye coordinate at the first time point; and a dominant eye determination unit configured to determine the dominant eye of the user with reference to the left-eye difference information and the right-eye difference information.

According to another aspect of the present disclosure, there is provided a system for determining a dominant eye, which includes: a coordinate acquisition unit configured to acquire indicator coordinates, left-eye coordinates, and right-eye coordinates of a user at a plurality of time points; a difference information calculation unit configured to calculate left-eye difference information about an angle, distance, or direction specified based on a straight line passing through the indicator coordinate at a first time point and the indicator coordinate at a second time point and a straight line passing through the indicator coordinate and the left-eye coordinate at the second time point, and right-eye difference information about an angle, distance, or direction specified based on a straight line passing through the indicator coordinate at the first time point and the indicator coordinate at the second time point and a straight line passing through the indicator coordinate and the right-eye coordinate at the second time point; and a dominant eye determination unit configured to determine the dominant eye of the user with reference to the left-eye difference information and the right-eye difference information.

In addition, there are further provided other methods and systems to implement the present disclosure, as well as non-transitory computer-readable recording media having stored thereon computer programs for executing the methods.

According to the present disclosure, it is possible to quickly and simply determine a dominant eye of a user by using coordinates of an indicator and both eyes of the user at a plurality of time points.

Furthermore, according to the present disclosure, it is possible to determine a dominant eye accurately by controlling a timing at which sensitive feedback associated with the dominant eye determination is provided.

### BRIEF DESCRIPTION OF THE DRAWINGS

FIG. 1 illustratively shows a schematic configuration of an overall system for determining a dominant-eye according to one embodiment of the present disclosure.

FIG. 2 illustratively shows an internal configuration of the dominant eye determination system in detail according to one embodiment of the present disclosure.

FIG. 3 illustratively shows a process of determining a dominant eye according to one embodiment of the present disclosure.

FIG. 3 illustratively shows a process of determining a dominant eye according to one embodiment of the present disclosure.

FIG. 4A illustratively shows a process of determining a dominant eye according to one embodiment of the present disclosure.

FIG. 4B illustratively shows a process of determining a dominant eye according to one embodiment of the present disclosure.

FIG. 5A illustratively shows a process of determining a dominant eye according to one embodiment of the present disclosure.

FIG. 5B illustratively shows a process of determining a dominant eye according to one embodiment of the present disclosure.

FIG. 6 illustratively shows a process of providing sensitive feedback associated with a dominant eye determination according to one embodiment of the present disclosure.

FIG. 7 illustratively shows sensitive feedback associated with a dominant eye determination according to one embodiment of the present disclosure.

FIG. 8A illustratively shows another process of determining the dominant eye according to one embodiment of the present disclosure.

FIG. 8B illustratively shows another process of determining the dominant eye according to one embodiment of the present disclosure.

### DETAILED DESCRIPTION

In the following detailed description of the present disclosure, references are made to the accompanying drawings that show, by way of illustration, specific embodiments in which the present disclosure may be practiced. These embodiments are described in sufficient detail to enable those skilled in the art to practice the present disclosure. It is to be understood that the various embodiments of the present disclosure, although different from each other, are not necessarily mutually exclusive. For example, specific shapes, structures, and features described herein may be implemented as modified from one embodiment to another without departing from the spirit and scope of the present disclosure. Furthermore, it shall be understood that the positions or arrangements of individual elements within each of the embodiments may also be modified without departing from the spirit and scope of the present disclosure. Therefore, the following detailed description is not to be taken in a limiting sense, and the scope of the present disclosure is to be taken as encompassing the scope of the appended claims and all equivalents thereof. In the drawings, like reference numerals refer to the same or similar elements throughout the several views.

Hereinafter, various preferred embodiments of the present disclosure will be described in detail with reference to the accompanying drawings to enable those skilled in the art to easily implement the present disclosure.

Configuration of Overall System

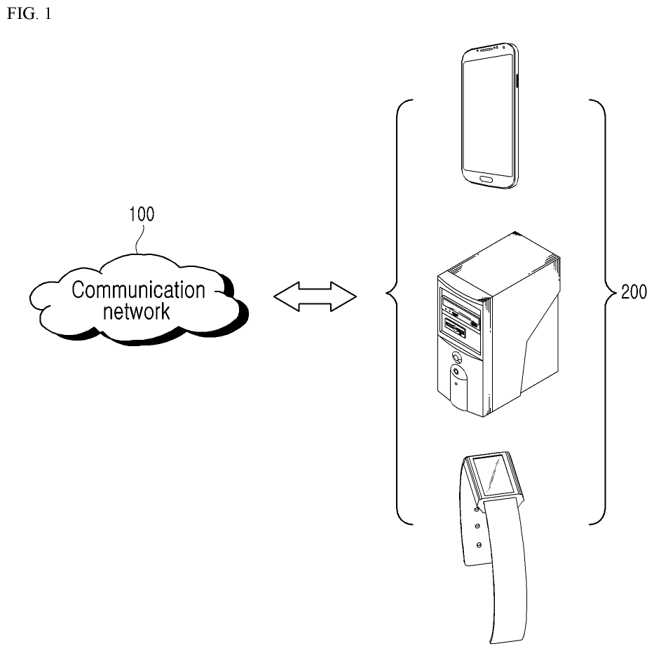

FIG. 1 illustratively shows a schematic configuration of an overall system for determining a dominant eye determination according to one embodiment of the present disclosure.

As illustrated in FIG. 1, the overall system according to one embodiment of the present disclosure may be configured to include a communication network 100 and a dominant eye management system 200.

First, the communication network 100 according to one embodiment of the present disclosure may be implemented by various communication modes including wired and wireless communications, and may include a variety of communication networks such as a local area network (LAN), a metropolitan area network (MAN), and a wide area network (WAN), and the like. Preferably, the communication network 100 described herein may be the Internet or the World Wide Web (WWW) which are well-known. However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as radio frequency (RF) communication, WiFi communication, cellular communication (e.g., Long Term Evolution (LTE) communication), Bluetooth communication (more specifically, Bluetooth Low Energy (BLE) communication), infrared communication, ultrasonic communication, or the like.

Next, the dominant eye management system 200 according to one embodiment of the present disclosure may be a digital device equipped with a memory means and a microprocessor provided for computing capabilities. The dominant eye management system 200 may be a server system.

According to one embodiment of the present disclosure, the dominant eye management system 200 may perform a function of: acquiring indicator coordinates, left-eye coordinates, and right-eye coordinates of a user at a plurality of time points; calculating left-eye difference information about an angle, distance, or direction specified based on a straight line passing through the indicator coordinate and the left-eye coordinate at a first time point among the plurality of time points and a straight line passing through the indicator coordinate and the left-eye coordinate at a second time point among the plurality of time points, and right-eye difference information about an angle, distance, or direction specified based on a straight line passing through the indicator coordinate and the right-eye coordinate at the first time point and a straight line passing through the indicator coordinate and the right-eye coordinate at the second time point; and determining a dominant eye of the user with reference to the left-eye difference information and the right-eye difference information.

Further, according to one embodiment of the present disclosure, the dominant eye management system 200 may perform a function of: acquiring indicator coordinates, left-eye coordinates, and right-eye coordinates of a user at a plurality of time points; calculating left-eye difference information about an angle, distance, or direction specified based on a straight line passing through the indicator coordinate at a first time point among the plurality of time points and the indicator coordinate at a second time point among the plurality of time points, and a straight line passing through the indicator coordinate and the left-eye coordinate at the first time point, and right-eye difference information about an angle, distance, or direction specified based on a straight line passing through the indicator coordinate at the first time point and the indicator coordinate at the second time point, and a straight line passing through the indicator coordinate and the right-eye coordinate at the first time point; and determining a dominant eye of the user with reference to the left-eye difference information and the right-eye difference information.

Furthermore, according to one embodiment of the present disclosure, the dominant eye management system 200 may perform a function of: acquiring indicator coordinates, left-eye coordinates, and right-eye coordinates of a user at a plurality of time points; calculating left-eye difference information about an angle, distance, or direction specified based on a straight line passing through the indicator coordinate at a first time point among the plurality of the time points and the indicator coordinate at a second time point among the plurality of the time points, and a straight line passing through the indicator coordinate and the left-eye coordinate at the second time point, and right-eye difference information about an angle, distance, or direction specified based on a straight line passing through the indicator coordinate at the first time point and the indicator coordinate at the second time point, and a straight line passing through the indicator coordinate and the right-eye coordinate at the second time point; and determining a dominant eye the user with reference to the left-eye difference information and the right-eye difference information.

The coordinates according to one embodiment of the present disclosure may be a concept that includes plane or spatial coordinates (e.g., rectangular coordinates, cylindrical coordinates, spherical coordinates) defined in a certain coordinate system which is used as a reference in the dominant eye management system 200 or a system associated with an imaging module (not shown) for obtaining an image of a user's body at a plurality of time points.

Further, the indicator according to one embodiment of the present disclosure may include various body portions such as hands, fingertips, fingers, feet, tiptoe, toes of the user. However, the present disclosure is not limited to the body portions described above. The indicator may be a concept that includes other various indication devices such as a pointer as long as the objects of the present disclosure may be achieved.

The configuration and functions of the dominant eye management system 200 according to the present disclosure will be described in more detail below. Meanwhile, although the dominant eye management system 200 has been described as above, such a description is illustrative and it will be apparent to those skilled in the art that at least a part of functions or elements required for the dominant eye management system 200 may be implemented or included in an external system (not shown), as necessary.

Configuration of Dominant Eye Management System

An internal configuration of the dominant eye management system 200 which performs an important function for implementing the present disclosure and functions of the respective elements thereof will be described below.

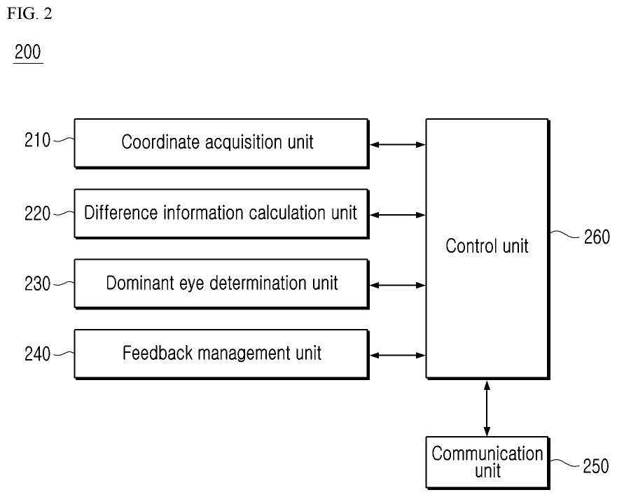

FIG. 2 illustratively shows a detailed internal configuration of the dominant eye management system 200 according to one embodiment of the present disclosure.

As illustrated in FIG. 2, the dominant eye management system 200 may be configured to include a coordinate acquisition unit 210, a difference information calculation unit 220, a dominant eye determination unit 230, a feedback management unit 240, and a communication unit 250, and a control unit 260. According to one embodiment of the present disclosure, at least some of the coordinate acquisition unit 210, the difference information calculation unit 220, the dominant eye determination unit 230, the feedback management unit 240, the communication unit 250 and the control unit 260 may be program modules to communicate with an external system. Such program modules may be included in the dominant eye management system 200 in the form of operating systems, application program modules, and other program modules, while they may be physically stored in a variety of commonly known storage devices. Further, the program modules may also be stored in a remote storage device that may communicate with the dominant eye management system 200. Further, such program modules may include, but not limited to, routines, subroutines, programs, objects, components, data structures, and the like for performing specific tasks or executing specific abstract data types as will be described below in accordance with the present disclosure.

The coordinate acquisition unit 210 according to one embodiment of the present disclosure may perform a function of acquiring indicator coordinates, left-eye coordinates, and right-eye coordinates of the user at a plurality of time points.

For example, the coordinate acquisition unit 210 according to one embodiment of the present disclosure may acquire two-dimensional or three-dimensional coordinates relating to fingertip coordinates, left-eye coordinates, and right-eye coordinates of the user, which are captured at the plurality of time points by at least one imaging module in a predetermined time unit. The aforementioned imaging module according to one embodiment of the present disclosure may be a module that is connected to the dominant eye management system 200 via the communication network 100, or included in the dominant eye management system 200, and may include a camera (including a two-dimensional or three-dimensional camera, an infrared camera, an ultraviolet camera, or the like), an image sensor, or the like.

The difference information calculation unit 220 according to one embodiment of the present disclosure may perform a function of calculating left-eye difference information about an angle, distance, or direction specified based on a straight line passing through an indicator coordinate and a left-eye coordinate at a first time point and a straight line passing through an indicator coordinate and a left-eye coordinate at a second time point, and right-eye difference information about an angle, distance, or direction specified based on a straight line passing through an indicator coordinate and a right-eye coordinate at the first time point and a straight line passing through an indicator coordinate and a right-eye coordinate at the second time point.

As an example, the difference information calculation unit 220 according to one embodiment of the present disclosure may calculate, as the left-eye difference information, a distance between a point at which the straight line passing through the indicator coordinate and the left-eye coordinate at the first time point intersects with a display region (not shown) representing an object to be controlled by the user, and a point at which the straight line passing through the indicator coordinate and the left-eye coordinate at the second time point intersects with the aforementioned display region. Further, the difference information calculation unit 220 according to one embodiment of the present disclosure may calculate, as the right-eye difference information, a distance between a point at which the straight line passing through the indicator coordinate and the right-eye coordinate at the first time point intersects with the aforementioned display region, and a point at which the straight line passing through the indicator coordinate and the right-eye coordinate at the second time point intersects with the aforementioned display region.

The display region according to one embodiment of the present disclosure may mean a region in which the object (e.g., an icon or the like) to be controlled by the user is displayed, and may be a concept that includes all regions defined by an intangible display, such as a screen display like a beam projector, a virtual display (e.g., a virtual plane) set with reference to an actual object or a virtual object placed on a three-dimensional space, or the like, in addition to a tangible display such as a liquid crystal display (LCD), a plasma display panel (PDP), a light emitting diode (LED), or an organic light emitting diode (OLED).

As another example, the difference information calculation unit 220 according to one embodiment of the present disclosure may calculate, as the left-eye difference information, a difference between an angle formed by the straight line passing through the indicator coordinate and the left-eye coordinate at the first time point and a first reference line, and an angle formed by the straight line passing through the indicator coordinate and the left-eye coordinate at the second time point and the aforementioned first reference line. Further, the difference information calculation unit 220 according to one embodiment of the present disclosure may calculate, as the right-eye difference information, a difference between an angle formed by the straight line passing through the indicator coordinate and the right-eye coordinate at the first time point and a second reference line, and an angle formed by the straight line passing through the indicator coordinate and the right-eye coordinate at the second time point and the aforementioned second reference line. According to one embodiment of the present disclosure, the aforementioned first reference line may be a line specified with reference to the left-eye coordinate and the aforementioned second reference line may be a line specified with reference to the right-eye coordinate. Further, according to one embodiment of the present disclosure, a reference line may not be divided into the first reference line and the second reference line described above, and the aforementioned angle may be calculated using merely one reference line (e.g., the line specified with reference to the left-eye coordinate, the line specified with reference to the right-eye coordinate, or a line connecting the left-eye coordinate and the right-eye coordinate).

As yet another example, the difference information calculation unit 220 according to one embodiment of the present disclosure may calculate, as the left-eye difference information, a difference between a direction in which the straight line passing through the indicator coordinate and the left-eye coordinate at the first time point is oriented and a direction in which the straight line passing through the indicator coordinate and the left-eye coordinate at the second time point is oriented. Further, the difference information calculation unit 220 according to one embodiment of the present disclosure may calculate, as the right-eye difference information, a difference between a direction in which the straight line passing through the indicator coordinate and the right-eye coordinate at the first time point is oriented and a direction in which the straight line passing through the indicator coordinate and the right-eye coordinate at the second time point is oriented. According to one embodiment of the present disclosure, the direction in which the aforementioned straight line is oriented, may be a direction (e.g., a direction angle, a rotational angle, or the like) specified based on a vector in which the left-eye coordinate or the right-eye coordinate is set as a start point and the indicator coordinate is set as an end point, a vector in which the indicator coordinate is set as a start point and the left-eye coordinate or the right-eye coordinate is set as an end point, a vector in which indicator coordinates at different time points are set as a start point and an end point, respectively (e.g., a vector in which the indicator coordinate at the first time point is set as a start point and the indicator coordinate at the second time point is set as an end point), or the like.

Further, the difference information calculation unit 220 according to one embodiment of the present disclosure may perform a function of calculating left-eye difference information about an angle, distance, or direction specified based on a straight line passing through an indicator coordinate at the first time point and an indicator coordinate at the second time point, and a straight line passing through the indicator coordinate and the left-eye coordinate at the first time point, and right-eye difference information about an angle, distance, or direction specified based on a straight line passing through the indicator coordinate at the first time point and the indicator coordinate at the second time point, and a straight line passing through the indicator coordinate and the right-eye coordinate at the first time point.

As an example, the difference information calculation unit 220 according to one embodiment of the present disclosure may calculate, as the left-eye difference information, a distance between a point at which the straight line passing through the indicator coordinate at the first time point and the indicator coordinate at the second time point intersects with a display region (not shown) in which an object to be controlled by the user is displayed, and a point at which the straight line passing through the indicator coordinate and the left-eye coordinate at the first time point intersects with the aforementioned display region. Further, the difference information calculation unit 220 according to one embodiment of the present disclosure may calculate, as the right-eye difference information, a distance between a point at which the straight line passing through the indicator coordinate at the first time point and the indicator coordinate at the second time point intersects with the aforementioned display region, and a point at which the straight line passing through the indicator coordinate and the right-eye coordinate at the first time point intersects with the aforementioned display region.

As another example, the difference information calculation unit 220 according to one embodiment of the present disclosure may calculate, as the left-eye difference information, a difference between an angle formed by the straight line passing through the indicator coordinate at the first time point and the indicator coordinate at the second time point and a first reference line, and an angle formed by the straight line passing through the indicator coordinate and the left-eye coordinate at the first time point and the aforementioned first reference line. Further, the difference information calculation unit 220 according to one embodiment of the present disclosure may calculate, as the right-eye difference information, a difference between an angle formed by the straight line passing through the indicator coordinate at the first time point and the indicator coordinate at the second time point and a second reference line, and an angle formed by the straight line passing through the indicator coordinate and the right-eye coordinate at the first time point and the aforementioned second reference line. According to one embodiment of the present disclosure, the aforementioned first reference line may be a line specified with reference to the left-eye coordinate and the aforementioned second reference line may be a line specified with reference to the right-eye coordinate. Further, according to one embodiment of the present disclosure, a reference line may not be divided into the first reference line and the second reference line described above, and the aforementioned angle may be calculated using merely one reference line (e.g., the line specified with reference to the left-eye coordinate, the line specified with reference to the right-eye coordinate, or a line connecting the left-eye coordinate and the right-eye coordinate).

As yet another example, the difference information calculation unit 220 according to one embodiment of the present disclosure may calculate, as the left-eye difference information, a difference between a direction in which the straight line passing through the indicator coordinate at the first time point and the indicator coordinate at the second time point is oriented and a direction in which the straight line passing through the indicator coordinate and the left-eye coordinate at the first time point is oriented. Further, the difference information calculation unit 220 according to one embodiment of the present disclosure may calculate, as the right-eye difference information, a difference between a direction in which the straight line passing through the indicator coordinate at the first time point and the indicator coordinate at the second time point is oriented and a direction in which the straight line passing through the indicator coordinate and the right-eye coordinate at the first time point is oriented.

Further, the difference information calculation unit 220 according to one embodiment of the present disclosure may perform a function of calculating left-eye difference information about an angle, distance, or direction specified based on the straight line passing through the indicator coordinate at the first time point and the indicator coordinate at the second time point, and the straight line passing through the indicator coordinate and the left-eye coordinate at the second time point, and right-eye difference information about an angle, distance, or direction specified based on the straight line passing through the indicator coordinate at the first time point and the indicator coordinate at the second time point, and the straight line passing through the indicator coordinate and the right-eye coordinate at the second time point.

As an example, the difference information calculation unit 220 according to one embodiment of the present disclosure may calculate, as the left-eye difference information, a distance between a point at which the straight line passing through the indicator coordinate at the first time point and the indicator coordinate at the second time point intersects with a display region (not shown) in which an object to be controlled by the user is displayed, and a point at which the straight line passing through the indicator coordinate and the left-eye coordinate at the second time point intersects with the aforementioned display region. Further, the difference information calculation unit 220 according to one embodiment of the present disclosure may calculate, as the right-eye difference information, a distance between a point at which the straight line passing through the indicator coordinate at the first time point and the indicator coordinate at the second time point intersects with the aforementioned display region, and a point at which the straight line passing through the indicator coordinate and the right-eye coordinate at the second time point intersects with the aforementioned display region.

As another example, the difference information calculation unit 220 according to one embodiment of the present disclosure may calculate, as the left-eye difference information, a difference between an angle formed by the straight line passing through the indicator coordinate at the first time point and the indicator coordinate at the second time point and a first reference line, and an angle formed by the straight line passing through the indicator coordinate and the left-eye coordinate at the second time point and the aforementioned first reference line. Further, the difference information calculation unit 220 according to one embodiment of the present disclosure may calculate, as the right-eye difference information, a difference between an angle formed by the straight line passing through the indicator coordinate at the first time point and the indicator coordinate at the second time point and a second reference line, and an angle formed by the straight line passing through the indicator coordinate and the right-eye coordinate at the second time point and the aforementioned second reference line. According to one embodiment of the present disclosure, the aforementioned first reference line may be a line specified with reference to the left-eye coordinate and the aforementioned second reference line may be a line specified with reference to the right-eye coordinate. Further, according to one embodiment of the present disclosure, a reference line may not be divided into the first reference line and the second reference line described above, and the aforementioned angle may be calculated using merely one reference line (e.g., the line specified with reference to the left-eye coordinate, the line specified with reference to the right-eye coordinate, or a line connecting the left-eye coordinate and the right-eye coordinate).

As yet another example, the difference information calculation unit 220 according to one embodiment of the present disclosure may calculate, as the left-eye difference information, a difference between a direction in which the straight line passing through the indicator coordinate at the first time point and the indicator coordinate at the second time point is oriented and a direction in which the straight line passing through the indicator coordinate and the left-eye coordinate at the second time point is oriented. Further, the difference information calculation unit 220 according to one embodiment of the present disclosure may calculate, as the right-eye difference information, a difference between a direction in which the straight line passing through the indicator coordinate at the first time point and the indicator coordinate at the second time point is oriented and a direction in which the straight line passing through the indicator coordinate and the right-eye coordinate at the second time point is oriented.

The dominant eye determination unit 230 according to one embodiment of the present disclosure may perform a function of determining the dominant eye of the user with reference to the left-eye difference information and the right-eye difference information described above.

As an example, the dominant eye determination unit 230 according to one embodiment of the present disclosure may determine, as the dominant eye, an eye associated with information having a smaller value of difference among the left-eye difference information and the right-eye difference information.

More specifically, in the case that the left-eye difference information corresponds to the distance between the point at which the straight line passing through the indicator coordinate and the left-eye coordinate at the first time point intersects with the display region (not shown) in which the object to be controlled by the user is displayed and the point at which the straight line passing through the indicator coordinate and the left-eye coordinate at the second time point intersects with the aforementioned display region, and the right-eye difference information corresponds to the distance between the point at which the straight line passing through the indicator coordinate and the right-eye coordinate at the first time point intersects with the aforementioned display region and the point at which the straight line passing through the indicator coordinate and the right-eye coordinate at the second time point intersects with the aforementioned display region, the dominant eye determination unit 230 according to one embodiment of the present disclosure may determine, as the dominant eye, an eye associated with information having a smaller value of distance difference among the left-eye difference information and the right-eye difference information.

Further, in the case that the left-eye difference information corresponds to the difference between the angle formed by the straight line passing through the indicator coordinate and the left-eye coordinate at the first time point and the first reference line (not shown) and the angle formed by the straight line passing through the indicator coordinate and the left-eye coordinate at the second time point and the aforementioned first reference line, and the right-eye difference information corresponds to the difference between the angle formed by the straight line passing through the indicator coordinate and the right-eye coordinate at the first time point and the second reference line (not shown) and the angle formed by the straight line passing through the indicator coordinate and the right-eye coordinate at the second time point and the aforementioned second reference, the dominant eye determination unit 230 according to one embodiment of the present disclosure may determine, as the dominant eye, an eye associated with information having a smaller value of angle difference among the left-eye difference information and the right-eye difference information.

Further, in the case that the left-eye difference information corresponds to the difference between the direction in which the straight line passing through the indicator coordinate and the left-eye coordinate at the first time point is oriented and the direction in which the straight line passing through the indicator coordinate and the left-eye coordinate at the second time point is oriented, and the right-eye difference information corresponds to the difference between the direction in which the straight line passing through the indicator coordinate and the right-eye coordinate at the first time point is oriented and the direction in which the straight line passing through the indicator coordinate and the right-eye coordinate at the second time point is oriented, the dominant eye determination unit 230 according to one embodiment of the present disclosure may determine, as the dominant eye, an eye associated with information having a smaller value of direction difference among the left-eye difference information and the right-eye difference information (e.g., an eye having a high degree of arrangement between the straight lines).

Further, in the case that the left-eye difference information corresponds to the distance between the point at which the straight line passing through the indicator coordinate at the first time point and the indicator coordinate at the second time point intersects with the display region (not shown) in which the object to be controlled by the user is displayed and the point at which the straight line passing through the indicator coordinate and the left-eye coordinate at the first time point intersects with the aforementioned display region, and the right-eye difference information corresponds to the distance between the point at which the straight line passing through the indicator coordinate at the first time point and the indicator coordinate at the second time point intersects with the aforementioned display region and the point at which the straight line passing through the indicator coordinate and the right-eye coordinate at the first time point intersects with the aforementioned display region, the dominant eye determination unit 230 according to one embodiment of the present disclosure may determine, as the dominant eye, an eye associated with information having a smaller value of distance difference among the left-eye difference information and the right-eye difference information.

Further, in the case that the left-eye difference information corresponds to the difference between the angle formed by the straight line passing through the indicator coordinate at the first time point and the indicator coordinate at the second time point and the first reference line (not shown) and the angle formed by the straight line passing through the indicator coordinate and the left-eye coordinate at the first time point and the aforementioned first reference line, and the right-eye difference information corresponds to the difference between the angle formed by the straight line passing through the indicator coordinate at the first time point and the indicator coordinate at the second time point and the second reference line (not shown) and the angle formed by the straight line passing through the indicator coordinate and the right-eye coordinate at the first time point and the aforementioned second reference line, the dominant eye determination unit 230 according to one embodiment of the present disclosure may determine, as the dominant eye, an eye associated with information having a smaller value of angle difference among the left-eye difference information and the right-eye difference information.

Further, in the case that the left-eye difference information corresponds to the difference between the direction in which the straight line passing through the indicator coordinate at the first time point and the indicator coordinate at the second time point is oriented and the direction in which the straight line passing through the indicator coordinate and the left-eye coordinate at the first time point is oriented, and the right-eye difference information corresponds to the difference between the direction in which the straight line passing through the indicator coordinate at the first time point and the indicator coordinate at the second time point is oriented and the direction in which the straight line passing through the indicator coordinate and the right-eye coordinate at the first time point is oriented, the dominant eye determination unit 230 according to one embodiment of the present disclosure may determine, as the dominant eye, an eye associated with information having a smaller value of direction difference among the left-eye difference information and the right-eye difference information (e.g., an eye having a high degree of accordance between the straight lines).

Further, in the case that the left-eye difference information corresponds to the distance between the point at which the straight line passing through the indicator coordinate at the first time point and the indicator coordinate at the second time point intersects with the display region (not shown) in which the object to be controlled by the user is displayed and the point at which the straight line passing through the indicator coordinate and the left-eye coordinate at the second time point intersects with the aforementioned display region, and the right-eye difference information corresponds to the distance between the point at which the straight line passing through the indicator coordinate at the first time point and the indicator coordinate at the second time point intersects with the aforementioned display region and the point at which the straight line passing through the indicator coordinate and the right-eye coordinate at the second time point intersects with the aforementioned display region, the dominant eye determination unit 230 according to one embodiment of the present disclosure may determine, as the dominant eye, an eye associated with information having a smaller value of distance difference among the left-eye difference information and the right-eye difference information.

Further, in the case that the left-eye difference information corresponds to the difference between the angle formed by and the angle formed by the straight line passing through the indicator coordinate at the first time point and the indicator coordinate at the second time point and the first reference line (not shown), and the angle formed by the straight line passing through the indicator coordinate and the left-eye coordinate at the second time point and the aforementioned first reference line corresponds to the left-eye difference information, and the right-eye difference information corresponds to the difference between the angle formed by the straight line passing through the indicator coordinate at the first time point and the indicator coordinate at the second time point and the second reference line (not shown), and the angle formed by the straight line passing through the indicator coordinate and the right-eye coordinate at the second time point and the aforementioned second reference line, the dominant eye determination unit 230 according to one embodiment of the present disclosure may determine, as the dominant eye, an eye associated with information having a smaller value of angle difference among the left-eye difference information and the right-eye difference information.

Further, in the case that the left-eye difference information corresponds to the difference between the direction in which the straight line passing through the indicator coordinate at the first time point and the indicator coordinate at the second time point is oriented, and the direction in which the straight line passing through the indicator coordinate and the left-eye coordinate at the second time point is oriented, and the right-eye difference information corresponds to the difference between the direction in which the straight line passing through the indicator coordinate at the first time point and the indicator coordinate at the second time point is oriented, and the direction in which the straight line passing through the indicator coordinate and the right-eye coordinate at the second time point is oriented c, the dominant eye determination unit 230 according to one embodiment of the present disclosure may determine, as the dominant eye, an eye associated with information having a smaller value of direction difference among the left-eye difference information and the right-eye difference information (e.g., an eye having a high degree of arrangement between the straight lines).

As another example, in the case that a difference between the left-eye difference information and the right-eye difference information is equal to or larger than a predetermined multiple (e.g., more than one), the dominant eye determination unit 230 according to one embodiment of the present disclosure may determine, as the dominant eye, an eye associated with information having a smaller value of difference among the left-eye difference information and the right-eye difference information.

More specifically, in the case that the left-eye difference information corresponds to the distance between the point at which the straight line passing through the indicator coordinate and the left-eye coordinate at the first time point intersects with the display region (not shown) in which the object to be controlled by the user is displayed, and the point at which the straight line passing through the indicator coordinate and the left-eye coordinate at the second time point intersects with the aforementioned display region, and the right-eye difference information corresponds to the distance between the point at which the straight line passing through the indicator coordinate and the right-eye coordinate at the first time point intersects with the aforementioned display region, and the point at which the straight line passing through the indicator coordinate and the right-eye coordinate at the second time point intersects with the aforementioned display region, if a distance difference between the left-eye difference information and the right-eye difference information is equal to or larger than the predetermined multiple (e.g., twice), then the dominant eye determination unit 230 may determine, as the dominant eye, an eye associated with information having a smaller value of distance difference among the left-eye difference information and the right-eye difference information.

Further, according to one embodiment of the present disclosure, in the case the that left-eye difference information corresponds to the difference between the angle formed by the straight line passing through the indicator coordinate and the left-eye coordinate at the first time point and the first reference line (not shown), and the angle formed by the straight line passing through the indicator coordinate and the left-eye coordinate at the second time point and the aforementioned first reference line, and the right-eye difference information corresponds to the difference between the angle formed by the straight line passing through the indicator coordinate and the right-eye coordinate at the first time point and the second reference line, and the angle formed by the straight line passing through the indicator coordinate and the right-eye coordinate at the second time point and the aforementioned second reference line, if an angle difference between the left-eye difference information and the right-eye difference information is equal to or larger than the predetermined multiple (e.g., twice), then the dominant eye determination unit 230 may determine, as the dominant eye, an eye associated with information having a smaller value of angle difference among the left-eye difference information and the right-eye difference information.

Further, according to one embodiment of the present disclosure, in the case that the left-eye difference information corresponds to the difference between the direction in which the straight line passing through the indicator coordinate and the left-eye coordinate at the first time point is oriented, and the direction in which the straight line passing through the indicator coordinate and the left-eye coordinate at the second time point is oriented, and the right-eye difference information corresponds to the difference between the direction in which the straight line passing through the indicator coordinate and the right-eye coordinate at the first time point is oriented, and the direction in which the straight line passing through the indicator coordinate and the right-eye coordinate at the second time point is oriented, if a direction difference between the left-eye difference information and the right-eye difference information is equal to or larger than the predetermined multiple (e.g., twice), then the dominant eye determination unit 230 may determine, as the dominant eye, an eye associated with information having a smaller value of direction difference among the left-eye difference information and the right-eye difference information.

Further, according to one embodiment of the present disclosure, in the case that the left-eye difference information corresponds to the distance between the point at which the straight line passing through the indicator coordinate at the first time point and the indicator coordinate at the second time point intersects with the display region (not shown) in which the object to be controlled by the user is displayed, and the point at which the straight line passing through the indicator coordinate and the left-eye coordinate at the first time point intersects with the aforementioned display region, and the right-eye difference information corresponds to the distance between the point at which the straight line passing through the indicator coordinate at the first time point and the indicator coordinate at the second time point intersects with the aforementioned display region, and the point at which the straight line passing through the indicator coordinate and the right-eye coordinate at the first time point intersects with the aforementioned display region, if a distance difference between the left-eye difference information and the right-eye difference information is equal to or larger than the predetermined multiple (e.g., twice), then the dominant eye determination unit 230 may determine, as the dominant eye, an eye associated with information having a smaller value of distance difference among the left-eye difference information and the right-eye difference information.

Further, according to one embodiment of the present disclosure, in the case that the left-eye difference information corresponds to the difference between the angle formed by the straight line passing through the indicator coordinate at the first time point and the indicator coordinate at the second time point and the first reference line (not shown), and the angle formed by the straight line passing through the indicator coordinate and the left-eye coordinate at the first time point and the aforementioned first reference line, and the right-eye difference information corresponds to the difference between the angle formed by the straight line passing through the indicator coordinate at the first time point and the indicator coordinate at the second time point and the second reference line (not shown), and the angle formed by the straight line passing through the indicator coordinate and the right-eye coordinate at the first time point and the aforementioned second reference line, if an angle difference between the left-eye difference information and the right-eye difference information is equal to or larger than the predetermined multiple (e.g., twice), then the dominant eye determination unit 230 may determine, as the dominant eye, an eye associated with information having a smaller value of angle difference among the left-eye difference information and the right-eye difference information.

Further, according to one embodiment of the present disclosure, in the case that the left-eye difference information corresponds to the difference between the direction in which the straight line passing through the indicator coordinate at the first time point and the indicator coordinate at the second time point is oriented, and the direction in which the straight line passing through the indicator coordinate and the left-eye coordinate at the first time point is oriented, and the right-eye difference information corresponds to the difference between the direction in which the straight line passing through the indicator coordinate at the first time point and the indicator coordinate at the second time point is oriented, and the direction in which the straight line passing through the indicator coordinate and the right-eye coordinate at the first time point is oriented, if a direction (e.g., a direction angle) difference between the left-eye difference information and the right-eye difference information is equal to or larger than the predetermined multiple (e.g., twice), then the dominant eye determination unit 230 may determine, as the dominant eye, an eye associated with information having a smaller value of direction difference among the left-eye difference information and the right-eye difference information.

Further, according to one embodiment of the present disclosure, in the case that the left-eye difference information corresponds to the distance between the point at which the straight line passing through the indicator coordinate at the first time point and the indicator coordinate at the second time point intersects with the display region (not shown) in which the object to be controlled by the user is displayed, and the point at which the straight line passing through the indicator coordinate and the left-eye coordinate at the second time point intersects with the aforementioned display region, and the right-eye difference information corresponds to the distance between the point at which the straight line passing through the indicator coordinate at the first time point and the indicator coordinate at the second time point intersect the aforementioned display region, and the point at which the straight line passing through the indicator coordinate and the right-eye coordinate at the second time point intersects with the aforementioned display region, if a distance difference between the left-eye difference information and the right-eye difference information is equal to or larger than the predetermined multiple (e.g., twice), then the dominant eye determination unit 230 may determine, as the dominant eye, an eye associated with information having a smaller value of distance difference among the left-eye difference information and the right-eye difference information.

Further, according to one embodiment of the present disclosure, in the case that the left-eye difference information corresponds to the difference between the angle formed by the straight line passing through the indicator coordinate at the first time point and the indicator coordinate at the second time point and the first reference line (not shown), and the angle formed by the straight line passing through the indicator coordinate and the left-eye coordinate at the second time point and the aforementioned first reference line, and the right-eye difference information corresponds to the difference between the angle formed by the straight line passing through the indicator coordinate at the first time point and the indicator coordinate at the second time point and the second reference line (not shown), and the angle formed by the straight line passing through the indicator coordinate and the right-eye coordinate at the second time point and the aforementioned second reference line, if an angle difference between the left-eye difference information and the right-eye difference information is equal to or larger than the predetermined multiple (e.g., twice), then the dominant eye determination unit 230 may determine, as the dominant eye, an eye associated with information having a smaller value of angle difference among the left-eye difference information and the right-eye difference information.

Further, according to one embodiment of the present disclosure, in the case that the left-eye difference information corresponds to the difference between the direction in which the straight line passing through the indicator coordinate at the first time point and the indicator coordinate at the second time point is oriented, and the direction in which the straight line passing through the indicator coordinate and the left-eye coordinate at the second time point is oriented, and the right-eye difference information corresponds to the difference between the direction in which the straight line passing through the indicator coordinate at the first time point and the indicator coordinate at the second time point is oriented, and the direction in which the straight line passing through the indicator coordinate and the right-eye coordinate at the second time point is oriented, if a direction (e.g., a direction angle) difference between the left-eye difference information and the right-eye difference information is equal to or larger than the predetermined multiple (e.g., twice), then the dominant eye determination unit 230 may determine, as the dominant eye, an eye associated with information having a smaller value of direction difference among the left-eye difference information and the right-eye difference information.

The dominant eye determination unit 230 according to one embodiment of the present disclosure may also determine information about the predetermined multiple for each user.

As an example, the dominant eye determination unit 230 according to one embodiment of the present disclosure may determine the predetermined multiple with reference to a user's profile (e.g., height, hand size, age, or the like).

As another example, the dominant eye determination unit 230 may determine the predetermined multiple with reference to a previously applied (or another) dominant eye determination pattern for a user, or a dominant eye determination pattern applied for another user having a similar profile to the respective user.

The feedback management unit 240 according to one embodiment of the present disclosure may perform a function of providing sensitive feedback associated with the dominant eye determination to the user when a distance between a point specified based on an indicator coordinate and the left-eye coordinate, and a point specified based on the indicator coordinate and a right-eye coordinate is within a predetermined level.

As an example, when a distance between a point at which a straight line passing through the indicator coordinate and the left-eye coordinate intersects a display region in which an object to be controlled by the user is displayed, and a point at which a straight line passing through the indicator coordinate and the right-eye coordinate intersects with the aforementioned display region is within the predetermined level, the feedback management unit 240 according to one embodiment of the present disclosure may provide visual feedback (e.g., a virtual touch button) associated with the dominant eye determination to the user. Further, when both the point specified based on the indicator coordinate and the left-eye coordinate and the point specified based on the indicator coordinate and the right-eye coordinate exist in a predetermined area within the display region in which the object to be controlled by the user is displayed, the feedback management unit 240 according to one embodiment of the present disclosure may provide the sensitive feedback associated with the dominant eye determination to the user.

As an example, when both the point at which the straight line passing through the indicator coordinate and the left-eye coordinate intersects with the display region in which the object to be controlled by the user is displayed, and the point at which the straight line passing through the indicator coordinate and the right-eye coordinate intersects with the aforementioned display region, exist in a predetermined area within the aforementioned display region, the feedback management unit 240 according to one embodiment of the present disclosure may provide the visual feedback (e.g., the virtual touch button) associated with the dominant eye determination to the user.

The communication unit 250 according to one embodiment of the present disclosure may perform a function to enable data transmission and reception from/to the coordinate acquisition unit 210, the difference information calculation unit 220, the dominant eye determination unit 230, and the feedback management unit 240.

The control unit 260 according to one embodiment of the present disclosure may perform a function to control data flow among the coordinate acquisition unit 210, the difference information calculation unit 220, the dominant eye determination unit 230, the feedback management unit 240, and the communication unit 250. That is, the control unit 260 according to the present disclosure may control data flow into/out of the dominant eye management system 200, or data flow among the respective components of the dominant eye management system 200, such that the coordinate acquisition unit 210, the difference information calculation unit 220, the dominant eye determination unit 230, the feedback management unit 240, and the communication unit 250 execute carry out their particular functions, respectively.

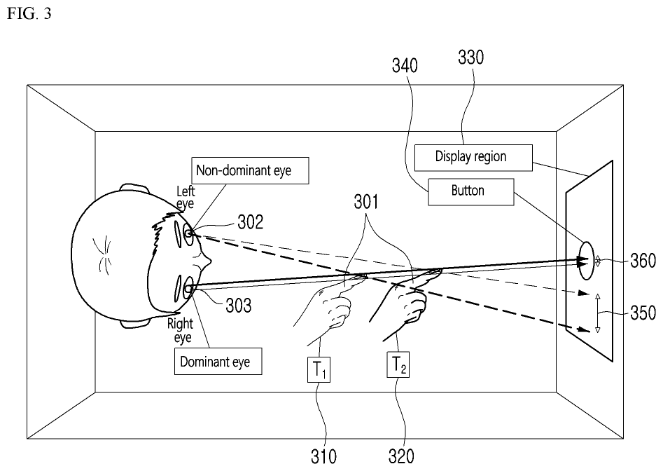

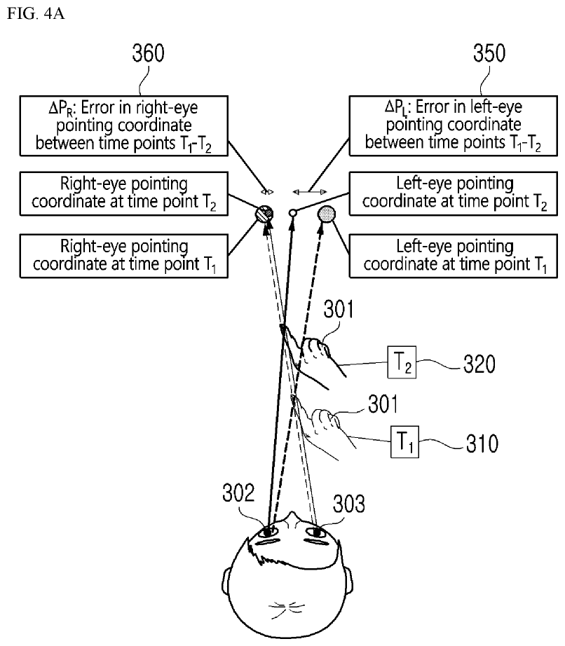

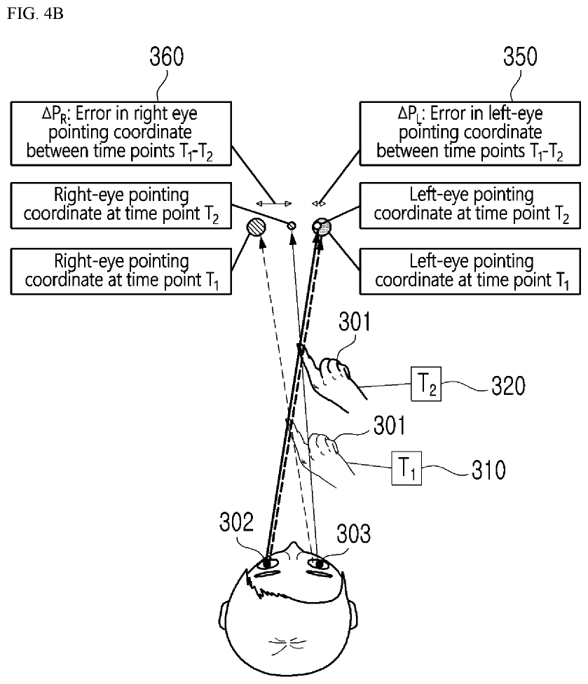

FIGS. 3 and 4 illustratively show a process of calculating the dominant eye according to one embodiment of the present disclosure.

First, referring to FIGS. 3 and 4, at each of a first time point 310 and a second time point 320 according to one embodiment of the present disclosure, the user may take a posture that matches his/her eyes and indicator with a virtual button 340 within a display region 330 according to a predetermined instruction.

Then, according to one embodiment of the present disclosure, an indicator coordinate 301, a left-eye coordinate 302, and a right-eye coordinate 303 of the user at each of the first time point 310 and the second time point 320 may be acquired.

Subsequently, according to one embodiment of the present disclosure, left-eye difference information 350 about a distance specified based on a straight line passing through the indicator coordinate 301 and the left-eye coordinate 302 at the first time point 310 and a straight line passing through the indicator coordinate 301 and the left-eye coordinate 302 at the second time point 320, and right-eye difference information 360 about a distance specified based on a straight line passing through the indicator coordinate 301 and the right-eye coordinate 303 at the first time point 310 and a straight line passing through the indicator coordinate 301 and the right-eye coordinate 303 at the second time point 320 may be calculated.

According to one embodiment of the present disclosure, left-eye difference information about a direction specified based on the straight line (hereinafter, a first straight line) passing through the indicator coordinate 301 and the left-eye coordinate 302 at the first time point 310 and the straight line (hereinafter, a second straight line) passing through the indicator coordinate 301 and the left-eye coordinate 302 at the second time point 320, and right-eye difference information about a direction specified based on the straight line (hereinafter, a third straight line) passing through the indicator coordinate 301 and the right-eye coordinate 303 at the first time point 310 and the straight line (hereinafter, a fourth straight line) passing through the indicator coordinate 301 and the right-eye coordinate 303 at the second time point 320 may be calculated. In this case, the left-eye difference information may mean a difference between directions in which the first straight line and the second straight line are oriented (e.g., such an extent that the first straight line and the second straight line are accorded to each other or similar direction angles to each other). The right-eye difference information may mean a difference between directions in which the third straight line and the fourth straight line are oriented (e.g., such an extent that the third straight line and the fourth straight line are accorded to each other or similar direction angles to each other).

Thereafter, according to one embodiment of the present disclosure, the dominant eye of the user may be determined based on the left-eye difference information 350 and the right-eye difference information 360.

As an example, according to one embodiment of the present disclosure, an eye associated with information having a smaller value of distance difference among the left-eye difference information 350 and the right-eye difference information 360 may be determined as the dominant eye. That is, in the case of FIGS. 3 and 4A, the right eye may be determined as the dominant eye (on the other hand, the right eye having a high degree of accordance between the directions in which the straight lines are oriented in the left-eye difference information or the right-eye difference information associated with the direction described above, may be determined as the dominant eye), and in the case of FIG. 4B, the left eye may be determined as the dominant eye.

As another example, according to one embodiment of the present disclosure, in the case that the left-eye difference information 350 and the right-eye difference information 360 are different from each other by a predetermined multiple or more (e.g., when the left-eye difference information 350 is different from the right-eye difference information 360 by five times or more), an eye associated with information having a smaller value of distance difference among the left-eye difference information 350 and the right-eye difference information 360 may be determined as the dominant eye. That is, in the case of FIGS. 3 and 4A, the right eye may be determined as the dominant eye, and in the case of FIG. 4B, the left eye may be determined as the dominant eye.

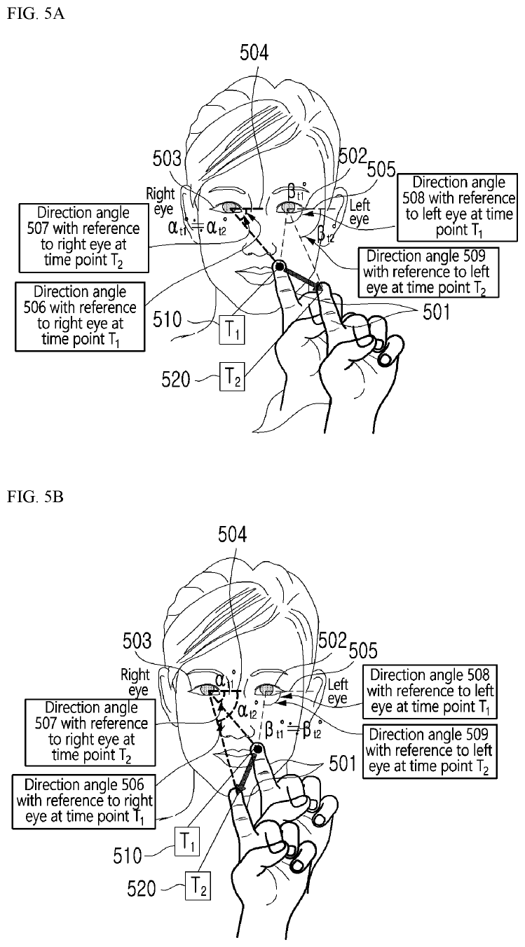

FIGS. 5A and 5B illustratively show another process of determining the dominant eye according to one embodiment of the present disclosure.

First, referring to FIGS. 5A and 5B, according to one embodiment of the present disclosure, at each of a first time point 510 and a second time point 520, the user may take a posture that matches his/her eyes and indicator with a virtual button (not shown) within a display region (not shown) according to a predetermined instruction.

Then, according to one embodiment of the present disclosure, an indicator coordinate 501, a left-eye coordinate 502, and a right-eye coordinate 503 of the user at each of the first time point 510 and the second time point 520 may be acquired.

Subsequently, according to one embodiment of the present disclosure, left-eye difference information about a difference between an angle 508 formed by a straight line passing through the indicator coordinate 501 and the left-eye coordinate 502 at the first time point 510 and a first reference line 505, and an angle 509 formed by a straight line passing through the indicator coordinate 501 and the left-eye coordinate 502 at the second time point 520 and the first reference line 505, and right-eye difference information about a difference between an angle 506 formed by a straight line passing through the indicator coordinate 501 and the right-eye coordinate 503 at the first time point 510 and a second reference line 504, and an angle 507 formed by a straight line passing through the indicator coordinate 501 and the right-eye coordinate 503 at the second time point 520 and the second reference line 504, may be calculated.

Thereafter, according to one embodiment of the present disclosure, the dominant eye of the user may be determined based on the left-eye difference information and the right-eye difference information described above.

As an example, according to one embodiment of the present disclosure, an eye associated with information having a smaller value of angle difference among the left-eye difference information and the right-eye difference information may be determined as the dominant eye. That is, in the case of FIG. 5A, the right eye may be determined as the dominant eye, and in the case of FIG. 5B, the left eye may be determined as the dominant eye.

As another example, according to one embodiment of the present disclosure, in the case that the left-eye difference information and the right-eye difference information are different from each other by a predetermined multiple or more (e.g., when the left-eye difference information is different from the right-eye difference information by five times or more), an eye associated with information having a smaller value of angle difference among the left-eye difference information and the right-eye difference information may be determined as the dominant eye. That is, in the case of FIG. 5A, the right eye may be determined as the dominant eye, and in the case of FIG. 5B, the left eye may be determined as the dominant eye.

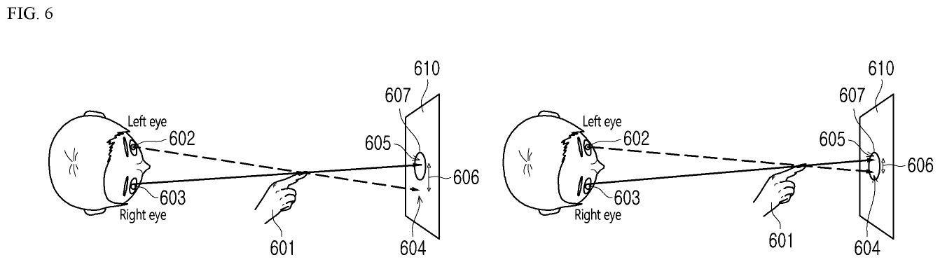

FIG. 6 illustratively shows a process of providing sensitive feedback associated with dominant eye determination according to one embodiment of the present disclosure.

First, referring to FIG. 6, according to one embodiment of the present disclosure, when a distance 606 between a point 604 specified based on an indicator coordinate 601 and a left-eye coordinate 602, and a point 605 specified based on the indicator coordinate 601 and a right-eye coordinate 603 is within a predetermined level, the sensitive feedback associated with the dominant eye determination may be provided to the user.

As an example, according to one embodiment of the present disclosure, when the distance 606 between the point 604 at which a straight line passing through the indicator coordinate 601 and the left-eye coordinate 602 intersects with a display region 610 in which an object to be controlled by the user is displayed, and the point 605 at which a straight line passing through the indicator coordinate 601 and the right-eye coordinate 603 intersects with the display region 610 is within the predetermined level (for example, 10 cm), a visual feedback (e.g., a virtual touch button) associated with the dominant eye determination may be provided to the user.

Further, according to one embodiment of the present disclosure, when both the point 604 specified based on the indicator coordinate 601 and the left-eye coordinate 602 and the point 605 specified based on the indicator coordinate 601 and the right-eye coordinate 603 exist in a predetermined area 607 within the display region 610 in which the object to be controlled by the user is displayed, the sensitive feedback associated with the dominant eye determination may be provided to the user.

As an example, according to one embodiment of the present disclosure, when both the point 604 at which a straight line passing through the indicator coordinate 601 and the left-eye coordinate 602 intersects with the display region 610 in which the object to be controlled by the user is displayed, and the point 605 at which a straight line passing through the indicator coordinate 601 and the right-eye coordinate 603 intersects with the display region 610 exist in the predetermined area 607 within the display region 610, the visual feedback (e.g., the virtual touch button) associated with the dominant eye determination may be provided to the user.

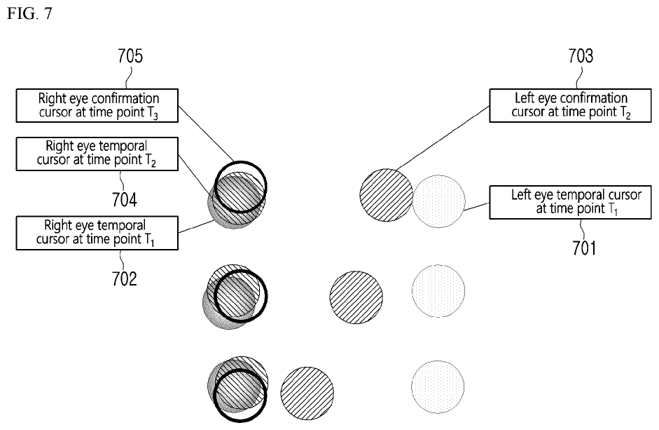

FIG. 7 illustratively shows sensitive feedback associated with the dominant eye determination according to one embodiment of the present disclosure.

Referring to FIG. 7, according to one embodiment of the present disclosure, in a situation in which visual feedback is provided as an example of the sensitive feedback on a display region (not shown), both the visual feedbacks (i.e., left-eye temporary cursors) 701 and 703 corresponding to the left eye, and the visual feedbacks (i.e., right-eye temporary cursors) 702 and 704 corresponding to the right eye may continue to be displayed until the dominant eye of the user is determined. Further, according to one embodiment of the present disclosure, after the dominant eye of the user is determined, merely a visual feedback 705 (i.e., right-eye confirmation cursor) corresponding to the right eye among the visual feedbacks corresponding to the left eye (i.e., left-eye temporary cursors) and the visual feedbacks corresponding to the right eye (i.e., right-eye temporary cursors) may be displayed.

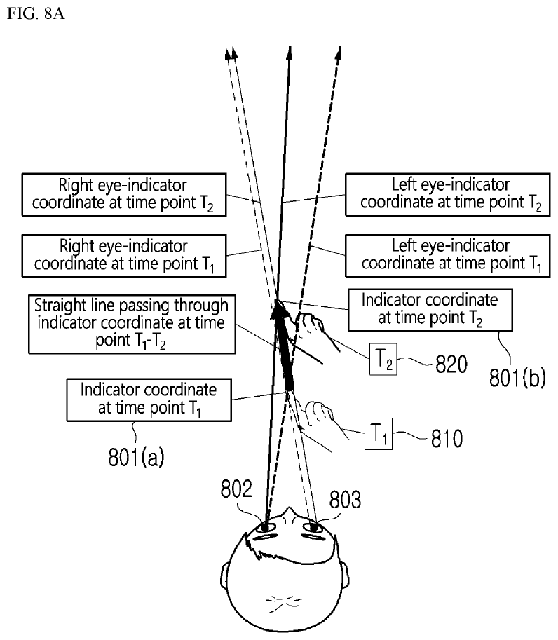

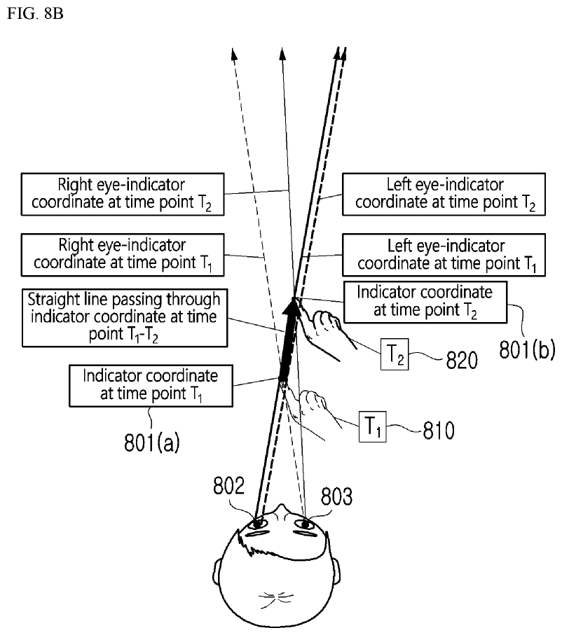

FIGS. 8A and 8B illustratively show another process of determining the dominant eye according to one embodiment of the present disclosure.

First, referring to FIGS. 8A and 8B, according to one embodiment of the present disclosure, at each of a first time point 810 and a second time point 820, the user may take a posture that matches his/her eyes and indicator with a virtual button (not shown) within a display region (not shown) according to a predetermined instruction.

Then, according to one embodiment of the present disclosure, indicator coordinates 801(a) and 801(b), a left-eye coordinate 802, and a right-eye coordinate 803 of the user at each of the first time point 810 and the second time point 820 may be acquired.

Subsequently, according to one embodiment of the present disclosure, left-eye difference information about a distance specified based on a straight line passing through the indicator coordinate 801a at the first time point 810 and the indicator coordinate 801b at the second time point 820, and a straight line passing through the indicator coordinate 801a and the left-eye coordinate 802 at the first time point 810, and right-eye difference information about a distance specified based on the straight line passing through the indicator coordinate 801a at the first time point 810 and the indicator coordinate 801b at the second time point 820, and a straight line passing through the indicator coordinate 801a and the right-eye coordinate 803 at the first time point 810, may be calculated. Further, according to one embodiment of the present disclosure, left-eye difference information about a distance specified based on the straight line passing through the indicator coordinate 801a at the first time point 810 and the indicator coordinate 801b at the second time point 820, and a straight line passing through the indicator coordinate 801b and the left-eye coordinate 802 at the second time point 820, and right-eye difference information about a distance specified based on the straight line passing through the indicator coordinate 801a at the first time point 810 and the indicator coordinate 801b at the second time point 820, and a straight line passing through the indicator coordinate 801b and the right-eye coordinate 803 at the second time point 820, may be calculated.

According to one embodiment of the present disclosure, left-eye difference information about a direction specified based on a straight line (hereinafter, a fifth straight line) passing through the indicator coordinate 801a at the first time point 810 and the indicator coordinate 801b at the second time point 820, and a straight line (hereinafter, a sixth straight line) passing through the indicator coordinate 801a and the left-eye coordinate 802 at the first time point 810, and right-eye difference information about a direction specified based on the straight line (i.e., the fifth straight line) passing through the indicator coordinate 801a at the first time point 810 and the indicator coordinate 801b at the second time point 820, and a straight line (hereinafter, a seventh straight line) passing through the indicator coordinate 801a and the right-eye coordinate 803 at the first time point 810, may be calculated. In this case, the left-eye difference information may mean a difference between directions in which the fifth straight line and the sixth straight line are oriented (e.g., such an extent that the fifth straight line and the sixth straight line are accorded to each other or similar direction angles to each other). The right-eye difference information may mean a difference between directions in which the fifth straight line and the seventh straight line are oriented (e.g., such an extent that the fifth straight line and the seventh straight line are accorded to each other or similar direction angles to each other). Further, according to one embodiment of the present disclosure, left-eye difference information about a direction specified based on the straight line (i.e., the fifth straight line) passing through the indicator coordinate 801a at the first time point 810 and the indicator coordinate 801b at the second time point 820, and a straight line (hereinafter, an eighth straight line) passing through the indicator coordinate 801b and the left-eye coordinate 802 at the second time point 820, and right-eye difference information about a direction specified based on the straight line (i.e., the fifth straight line) passing through the indicator coordinate 801a at the first time point 810 and the indicator coordinate 801b at the second time point 820, and a straight line (hereinafter, a ninth straight line) passing through the indicator coordinate 801b and the right-eye coordinate 803 at the second time point 820, may be calculated. In this case, the left-eye difference information may mean a difference between directions in which the fifth straight line and the eighth straight line are oriented (e.g., such an extent that the fifth straight line and the eighth straight line are accorded to each other or similar direction angles to each other). The right-eye difference information may mean a difference between directions in which the fifth straight line and the ninth straight line are oriented (e.g., such an extent that the fifth straight line and the ninth straight line are accorded to each other or similar direction angles to each other).

Thereafter, according to one embodiment of the present disclosure, the dominant eye of the user may be determined based on the left-eye difference information and the right-eye difference information described above.

As an example, according to one embodiment of the present disclosure, an eye associated with information having a smaller value of distance difference among the left-eye difference information and the right-eye difference information may be determined as the dominant eye. That is, in the case of FIG. 8A, the right eye may be determined as the dominant eye (on the other hand, the right eye having a high degree of accordance between the directions in which the straight lines are oriented in the left-eye difference information or the right-eye difference information associated with the direction described above, may be determined as the dominant eye), and in the case of FIG. 8B, the left eye may be determined as the dominant eye.

As another example, according to one embodiment of the present disclosure, in the case that the left-eye difference information and the right-eye difference information are different from each other by a predetermined multiple or more (e.g., when the left-eye difference information is different from the right-eye difference information by five times or more), an eye associated with information having a smaller value of distance difference among the left-eye difference information and the right-eye difference information may be determined as the dominant eye. That is, in the case of FIG. 8A, the right eye may be determined as the dominant eye, and in the case of FIG. 8B, the left eye may be determined as the dominant eye.

The embodiments according to the present disclosure as described above may be implemented in the form of program instructions that can be executed by various computer components, and may be stored on a non-transitory computer-readable recording medium. The non-transitory computer-readable recording medium may include program instructions, data files, and data structures, separately or in combination. The program instructions stored on the non-transitory computer-readable recording medium may be specially designed and configured for the present disclosure, or may also be known and available to those skilled in the computer software field. Examples of the non-transitory computer-readable recording medium may include: magnetic media such as hard disks, floppy disks and magnetic tapes; optical media such as compact disk-read only memory (CD-ROM) and digital versatile disks (DVDs); magneto-optical media such as floptical disks; and hardware devices such as read only memory (ROM), random access memory (RAM) and flash memory, which are specially configured to store and execute program instructions. Examples of the program instructions may include not only machine language codes created by a compiler, but also high-level language codes that can be executed by a computer using an interpreter. The above hardware devices may be changed to one or more software modules to perform the processes of the present disclosure, and vice versa.

Although the present disclosure has been described above in terms of specific items such as detailed elements as well as the limited embodiments and the drawings, they are only provided to help more general understanding of the present disclosure, and the present disclosure is not limited to the above embodiments. It will be appreciated by those skilled in the art to which the present disclosure pertains that various modifications and changes may be made from the above description.

Therefore, the spirit of the present disclosure shall not be limited to the above-described embodiments, and the entire scope of the appended claims and their equivalents will fall within the scope and spirit of the present disclosure.

What is claimed is:

1. A method of determining a dominant eye, comprising the steps of:
acquiring indicator coordinates, left-eye coordinates, and right-eye coordinates of a user at a plurality of time points;
calculating left-eye difference information about an angle, distance, or direction specified based on a straight line passing through the indicator coordinate and the left-eye coordinate at a first time point and a straight line passing through the indicator coordinate and the left-eye coordinate at a second time point, and right-eye difference information about an angle, distance, or direction specified based on a straight line passing through the indicator coordinate and the right-eye coordinate at the first time point and a straight line passing through the indicator coordinate and the right-eye coordinate at the second time point; and
determining the dominant eye of the user with reference to the left-eye difference information and the right-eye difference information.

2. The method of claim 1, wherein the step of calculating the left-eye difference information and the right-eye difference information comprises:
calculating, as the left-eye difference information, a distance between a point at which the straight line passing through the indicator coordinate and the left-eye coordinate at the first time point intersects with a display region in which an object to be controlled by the user is displayed, and a point at which the straight line passing through the indicator coordinate and the left-eye coordinate at the second time point intersects with the display region; and
calculating, as the right-eye difference information, a distance between a point at which the straight line passing through the indicator coordinate and the right-eye coordinate at the first time point intersects with the display region, and a point at which the straight line passing through the indicator coordinate and the right-eye coordinate at the second time point intersects with the display region.

3. The method of claim 1, wherein the step of calculating the left-eye difference information and the right-eye difference information comprises:
calculating, as the left-eye difference information, a difference between an angle formed by the straight line passing through the indicator coordinate and the left-eye coordinate at the first time point and a first reference line, and an angle formed by the straight line passing through the indicator coordinate and the left-eye coordinate at the second time point and the first reference line; and
calculating, as the right-eye difference information, a difference between an angle formed by the straight line passing through the indicator coordinate and the right-eye coordinate at the first time point and a second reference line, and an angle formed by the straight line passing through the indicator coordinate and the right-eye coordinate at the second time point and the second reference line.

4. The method of claim 3, wherein the first reference line is specified with reference to the left-eye coordinate, and the second reference line is specified with reference to the right-eye coordinate.

5. The method of claim 1, wherein the step of calculating the left-eye difference information and the right-eye difference information comprises:
calculating, as the left-eye difference information, a difference between a direction in which the straight line passing through the indicator coordinate and the left-eye coordinate at the first time point is oriented, and a direction in which the straight line passing through the indicator coordinate and the left-eye coordinate at the second time point is oriented; and
calculating, as the right-eye difference information, a difference between a direction in which the straight line passing through the indicator coordinate and the right-eye coordinate at the first time point is oriented, and a direction in which the straight line passing through the indicator coordinate and the right-eye coordinate at the second time point is oriented.

6. The method of claim 5, wherein the direction in which the straight line is oriented is specified based on a vector having the left-eye coordinate or the right-eye coordinate as a starting point and the indicator coordinate as an end point.

7. The method of claim 1, wherein in the step of determining the dominant eye, an eye associated with information having a smaller value of difference among the left-eye difference information and the right-eye difference information is determined as the dominant eye.

8. The method of claim 1, wherein in the step of determining the dominant eye, when the left-eye difference information and the right-eye difference information are different from each other by a predetermined multiple or more, an eye associated with information having a smaller value of difference among the left-eye difference information and the right-eye difference information is determined as the dominant eye.

9. The method of claim 1, wherein, when a distance between a point specified based on the indicator coordinate and the left-eye coordinate and a point specified based on the indicator coordinate and the right-eye coordinate is within a predetermined level, a sensitive feedback associated with a dominant eye determination is provided to the user.

10. The method of claim 9, wherein, when a distance between a point at which the straight line passing through the indicator coordinate and the left-eye coordinate intersects with a display region in which an object to be controlled by the user is displayed, and a point at which the straight line passing through the indicator coordinate and the right-eye coordinate intersects with the display region is within the predetermined level, the sensitive feedback associated with the dominant eye determination is provided to the user.

11. The method of claim 1, wherein, when both a point at which the straight line passing through the indicator coordinate and the left-eye coordinate intersects with a display region in which an object to be controlled by the user is displayed, and a point at which the straight line passing through the indicator coordinate and the right-eye coordinate intersects with the display region exist in a predetermined area within the display region, a sensitive feedback associated with a dominant eye determination is provided to the user.

12. A method of determining a dominant eye, comprising the steps of:
acquiring indicator coordinates, left-eye coordinates, and right-eye coordinates of a user at a plurality of time points;
calculating left-eye difference information about an angle, distance, or direction specified based on a straight line passing through the indicator coordinate at a first time point and the indicator coordinate at a second time point, and a straight line passing through the indicator coordinate and the left-eye coordinate at the first time point, and right-eye difference information about an angle, distance, or direction specified based on a straight line passing through the indicator coordinate at the first time point and the indicator coordinate at the second time point, and a straight line passing through the indicator coordinate and the right-eye coordinate at the first time point; and
determining the dominant eye of the user with reference to the left-eye difference information and the right-eye difference information.

13. A non-transitory computer-readable recording medium having stored thereon a computer program for executing the method of claim 1.

14. A system for determining a dominant eye, comprising:
a coordinate acquisition unit configured to acquire indicator coordinates, left-eye coordinates, and right-eye coordinates of a user at a plurality of time points;
a difference information calculation unit configured to calculate left-eye difference information about an angle, distance, or direction specified based on a straight line passing through the indicator coordinate and the left-eye coordinate at a first time point and a straight line passing through the indicator coordinate and the left-eye coordinate at a second time point, and right-eye difference information about an angle, distance, or direction specified based on a straight line passing through the indicator coordinate and the right-eye coordinate at the first time point and a straight line passing through the indicator coordinate and the right-eye coordinate at the second time point; and
a dominant eye determination unit that determines the dominant eye of the user with reference to the left-eye difference information and the right-eye difference information.

15. A system for determining a dominant eye, comprising:
a coordinate acquisition unit configured to acquire indicator coordinates, left-eye coordinates, and right-eye coordinates of a user at a plurality of time points;
a difference information calculation unit configured to calculate left-eye difference information about an angle, distance, or direction specified based on a straight line passing through the indicator coordinate at a first time point and the indicator coordinate at a second time point and a straight line passing through the indicator coordinate and the left-eye coordinate at the first time point, and right-eye difference information about an angle, distance, or direction specified based on a straight line passing through the indicator coordinate at the first time point and the indicator coordinate at the second time point and a straight line passing through the indicator coordinate and the right-eye coordinate at the first time point; and
a dominant eye determination unit configured to determine the dominant eye of the user with reference to the left-eye difference information and the right-eye difference information.
